(19) United States
(12) Patent Application Publication
(10) Pub. No.: US 2002/0137111 A1
(43) Pub. Date: Sep. 26, 2002

(54) Chlamydia heat shock protein

(76) Inventor: Rosanna Wai-wan Peeling, Manitoba (CA)

Correspondence Address: Michael Williams, Ade & Company, 1700-360 Main Street, Winnipeg Manitoba, R3C 3Z3, CA

(21) Appl. No.: 09/759,272

(22) Filed: Jan. 16, 2001

(51) Int. Cl. G01N 33/554; G01N 33/569; C12P 21/04

(52) U.S. Cl. 435/7.32; 435/70.21

(57) ABSTRACT

An expression system for production of full-length Chlamydial heat shock protein 60 (CHSP60) and an immunoreactive subfragment thereof is described. The expression system is arranged such that the CHSP60 or fragment is initially isolated as a fusion protein which is then cleaved off, leaving purified CHSP60.

### FIELD OF THE INVENTION

The present invention relates generally to the field of immunoassays. More specifically, the present invention relates to recombinant proteins and their use in immunoassays for detecting the presence of antibodies to strains of Chlamydia.

### BACKGROUND OF THE INVENTION

Chlamydiae are obligate intracellular bacterial pathogens responsible for a wide range of infections in animals. The genus Chlamydia is divided into four species: C. trachomatis, C. pneumoniae, C. psittaci and C. pecorum. Chlamydia trachomatis infection is the most prevalent bacterial sexually-transmitted disease in many developed countries, including Canada. Women with cervical chlamydial infections are at risk of developing pelvic inflammatory disease, which can lead to long-term reproductive sequelae, such as chronic pelvic pain, ectopic pregnancy and tubal infertility. Chlamydia trachomatis is also the leading infectious cause of blindness and is estimated to affect 500 million people worldwide. Chlamydia pneumoniae is responsible for 10-20% of community-acquired pneumonia. Studies from around the world show that 40-60% of adult populations possess antibodies to C. pneumoniae, suggesting that infections and re-infections are quite common. Recent studies have linked persistent C. pneumoniae infection to a number of chronic diseases including atherosclerosis, asthma, exacerbation of chronic obstructive pulmonary disease, stroke, Alzheimer's disease and multiple sclerosis.

The invasion of chlamydiae into a human host creates a stressful condition in the host as well as a hostile environment for the chlamydiae, as the host mounts an immune response against the presence of the invading microbe. Thus, an up-regulation of the heat shock response frequently occurs in both chlamydiae and the host during an infection. Heat shock proteins are among the most abundant proteins in nature and are highly conserved amongst both eukaryotes and prokaryotes. The heat shock response is an important survival mechanism that safeguards the cell or microbe from conditions of stress. The response is triggered transcriptionally and results in the production of newly synthesized proteins within minutes of the cell or microbe encountering a stressful environment. Heat shock proteins are involved in vital cell functions, such as the assembly and disassembly of macromolecules.

The association between antibody response to a chlamydial heat shock protein and the development of tubal infertility was first shown by Brunham et al. , who reported that 11 of 13 chlamydia seropositive women with tubal infertility had antibody to a 57 kDa protein compared with 2 of 6 seropositive women with non-tubal causes of infertility and 1 of 11 seropositive pregnant controls (Brunham et al., 1985, J. Infect. Dis. 152: 1275-1282). This 57 kDa chlamydial protein was subsequently determined to belong to a group of proteins known as heat shock proteins (HSPs), specifically to the GroEL or HSP60 family. This family of proteins has amino acid sequences that are highly conserved among both prokaryotes and eukaryotes and has been implicated in the pathogenesis of immune diseases. The chlamydial Hsp60 (CHSP60) is constitutively expressed and its transcription is upregulated during conditions of stress. The protein is found in both forms of chlamydiae: the elementary body (EB), which is the extracellular, infectious form, and the reticulate body (RB), which is the intracellular, metabolically active form.

One of the hallmarks of chlamydial infection is that the symptoms are often mild or absent. Undiagnosed and untreated, the infection can persist in the body leading to chronic inflammation at the site of infection. The pathophysiology of these chronic disease conditions is thought to be immunologically mediated, and the CHSP60 has been implicated as a major antigen that stimulates the immunopathological response. In vitro studies of persistent infection show that CHSP60 is disproportionately expressed compared with other chlamydial proteins, such as the major outer membrane protein (Beatty et al, 1994, Infect Immun 62: 4059-4062). Studies in an animal model of pelvic inflammatory disease show that the CHSP60 antibody response is associated with the persistence of chlamydia in the fallopian tubes (Peeling et al, 1999, JID 180: 774-779). Thus, antibody response to CHSP60 is a marker for persistent chlamydial infections and as such can be used to predict the risk of developing long term complications as a result of prior chlamydial infections.

Studies of human chlamydial infection have shown that antibody response to the 60 kDa chlamydial heat shock protein, CHSP60, is associated with the development of adverse sequelae following ocular and genital chlamydial infection with C. trachomatis (Peeling and Mabey, 1999, Infect. Dis. Obstet. Gynecol. 7:72-79). More recently, the presence of CHSP60 antibody was reported to be correlated with long-term sequelae of C. pneumoniae infection such as atherosclerosis and asthma (Fong et al 2000, manuscript submitted to Clinical Infectious Diseases; Peeling and Hahn, unpublished data). However, the mechanism by which CHSP60 contributes to the immunopathological sequelae associated with chlamydial infection remains unclear. It is speculated that antibody response to CHSP60 may be a marker of persistent chlamydial infection or of an autoimmune response elicited as a result of molecular mimicry with human HSP60. CHSP-60 has been localized in human atheroma (Kol et al, 1998, Circulation 98: 300-307), and may play a role in atherogenesis by regulation of macrophage tumor necrosis factor-alpha (TNFα) and matrix metalloproteinase expression.

Since the tubal infertility study by Brunham et al. (Brunham et al., 1985) numerous groups have examined the relationship between antibody to CHSP60 and adverse reproductive sequelae associated with C. trachomatis infection. Wager et al. showed by immunoblot that 6 (31%) of 19 patients with pelvic inflammatory disease and 17 (81%) of 21 ectopic pregnancy patients had antibody to CHSP60 in a Triton X-100 soluble extract (Wager et al., 1990, J. Infect Dis. 162: 922-927). However, the presence of a chlamydia structural protein with molecular weight very similar to CHSP60 makes immunoblot reactivity of CHSP60 difficult to interpret, especially for low titer sera. To overcome this problem, Brunham et al. used a sarcosyl-soluble extract of C. trachomatis (the structural protein is sarkosyl insoluble) to enrich for CHSP60. Nineteen (91%) of 21 seropositive patients with ectopic pregnancy had antibody to this enriched CHSP60 extract by immunoblot compared to 25% of controls (Brunham et al., 1992, J. Infect. Dis. 165: 1076-1081).

These previous studies used whole Chlamydia organisms or enriched semi-purified protein preparations from chlamydia to correlate human immune responses to chlamydial antigens with reproductive sequelae. There are a number of drawbacks associated with the use of such preparations. Specifically, these preparations are not pure, but contain many other contaminating chlamydial proteins in addition to CHSP60. Therefore, serum antibody responses to these preparations can only be analyzed by SDS-polyacrylamide gel electrophoresis and immunoblotting, to confirm that the antibody is directed to the CHSP60 and not one of the contaminating proteins or LPS. This method of analyzing antibody response to the CHSP60 is extremely laborious and time-consuming. In addition, pure chlamydial organisms are required for these preparations which requires tissue culture for the growth of the chlamydiae, which is both time-consuming and costly. Hence all these studies on pelvic inflammatory disease, ectopic pregnancy, and tubal infertility were limited by small sample size and adequate seropositive fertile controls.

To obtain large quantities of CHSP60, Cerrone et al cloned the gene for CHSP60 and expressed the CHSP60 and fragments of CHSP60 as fusion proteins linked, at its N terminal, to a 26 kDa glutathione-S-transferase from Schistosoma japonicum. Using this recombinant CHSP60 and sera from 5 women with pelvic inflammatory disease or ectopic pregnancy, Cerrone et al. confirmed that immune response detected in these studies was to this protein (Cerrone et al., 1991, Infect. Immun. 59: 79-90). Cerrone et al reported that sera from C. trachomatis infected patients reacted with the fusion protein containing amino acids 274-402 and 405-544 of CHSP-60, but not with those containing amino acids 1-51, 50-143 and 50-266 of CHSP-60. As will be appreciated by one knowledgeable in the art, this indicates that the immunoreactivity of specific fragments of CHSP60 cannot be predicted in advance, likely due to protein folding affecting presentation of antigenic domains. Furthermore, since the recombinant CHSP60 comprised a large fusion partner (26 kDa glutathione-S-transferase) which is similar in size or larger than fragments of CHSP60, it is also possible that the GST tag is blocking or masking antigenic determinates at the amino terminus of the CHSP60 fusions. It is of note that the GST tag can be cleaved from the fusion protein by treatment with thrombin; however, thrombin also recognizes sites within the peptide of interest (in this case, CHSP60), meaning that removal of the GST tag may result in the entire fusion protein being cleaved at multiple sites.

In addition, Yuan et al (Yuan et al., 1992, Infect Immun 60: 2288-2296) describe the construction of lacZ-CHSP60 fusion peptides which were used to generate monoclonal antibodies. The monoclonal antibodies were subsequently mapped to epitopes at amino acids 8-14 and 177-189 of CHSP60. It is of note that these epitopes are present in fusion peptides which failed to react with patient sera when tested by Cerrone et al, as discussed above.

To further examine the role of CHSP60 in tubal infertility and to facilitate the study of a larger number of samples, we developed an ELISA using a full-length CHSP60 GST fusion peptide as antigen. In a study by Toye et al., this ELISA assay was used to determine the prevalence of antibody to the CHSP60 in women with tubal infertility. Antibody to C. trachomatis was present in 32 (72.7%) of 44 of women with tubal infertility compared with 9 (32.1%) of 28 with other causes of infertility and 55 (28.9%) of 190 pregnant women (p<0.001). The CHSP60 ELISA detected Chlamydia-associated tubal infertility in infertile women with a sensitivity of 81.3% and a specificity of 97.5% (Toye et al., 1993, J. Infect. Dis. 168: 1236-1240). Several other studies have been performed to demonstrate that there is a strong association between antibody response to the CHSP60 and the development of Chlamydia-associated tubal infertility (Peeling and Mabey, 1999, Dis. Obstet. Gynecol. 7: 72-79). It was also concluded that a CHSP60 ELISA may be useful as a predictor for poor fertility outcome (Claman et al., 1996, Fertil. Steril. 65: 146-149).

Since the CHSP60 ELISA is highly specific, it may prove useful in the investigation of infertile women as a marker of Chlamydia-associated tubal obstruction and lead to more selective use of invasive procedures (e.g. diagnostic laparosopy). This ELISA assay may also be used as a means of assessing the risk or presence of tubal obstruction in women seeking infertility treatment.

As described above, the clone used in the previous studies had a fusion partner, a 26 kDa glutathione-S-transferase, which necessitated the use of 2 parallel ELISA assays to assess the reactivity against the entire fusion protein as well as against the fusion partner. The use of the additional assays increases the time and labor required to carry out the ELISA assays, reducing the total number of samples that can be screened in a given period of time. Clearly, there is a need for a more efficient ELISA assay that would allow more samples to be processed and perhaps with higher sensitivity and specificity.

### SUMMARY OF THE INVENTION

According to a first aspect of the invention, there is provided a method of detecting anti-CHSP60 antibodies in a sample from a patient comprising:

providing purified CHSP60₈₀₋₂₇₇ or purified CHSP60₁₋₅₄₄;

binding the CHSP60₈₀₋₂₇₇ or CHSP60₁₋₅₄₄ to a support;

mixing the sample with the bound CHSP60₁₋₅₄₄ or CHSP60₈₀₋₂₇₇ under conditions such that anti-CHSP60 antibodies within the sample bind to the CHSP60₁₋₅₄₄ or CHSP60₈₀₋₂₇₇;

washing away unbound sample; and

detecting antibodies bound to the CHSP60₁₋₅₄₄ or CHSP60₈₀₋₂₇₇.

According to a second aspect of the invention, there is provided a kit comprising purified CHSP60₈₀₋₂₇₇. or CHSP60₁₋₅₄₄.

### BRIEF DESCRIPTION OF THE DRAWINGS

FIG. 1 shows the nucleotide sequence of the primers.

FIG. 2 shows the amino acid sequence of CHSP60.

FIG. 3 shows the sequence variance across amino acids 80-277 of CHSP60.

FIG. 4 is a schematic diagram of the expression system.

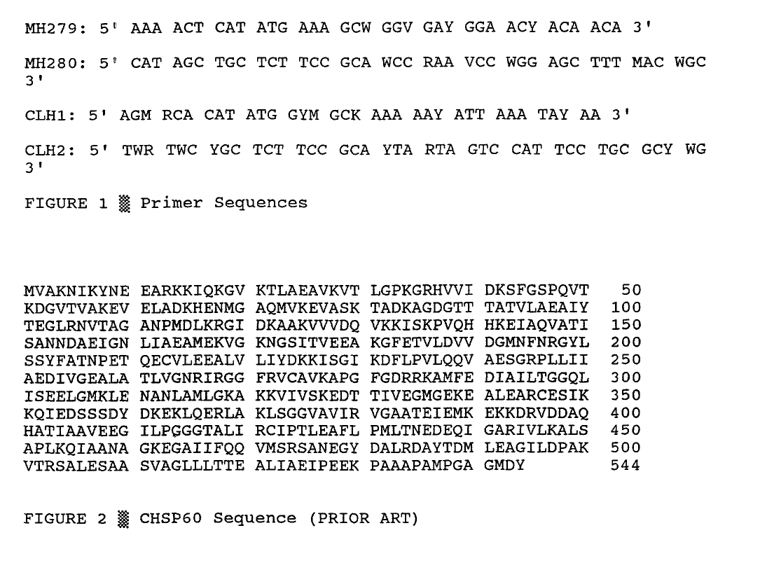

TABLE 1 shows absorbance readings at 405 nm for the CHSP60 ELISA assay for serum samples tested against serovar L₂ CHSP60-GST fusion and CHSP60₈₀₋₂₇₇.

TABLE 2 shows effectiveness of CHSP60₈₀₋₂₇₇ vs CHSP60-GST in asthma cases.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

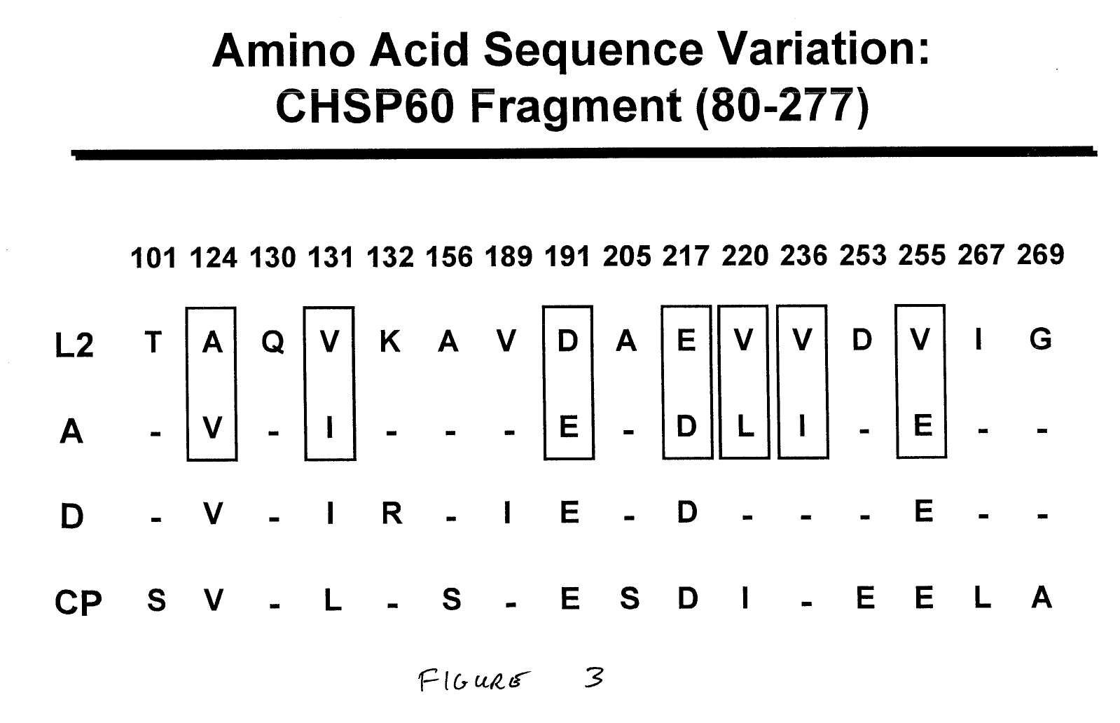

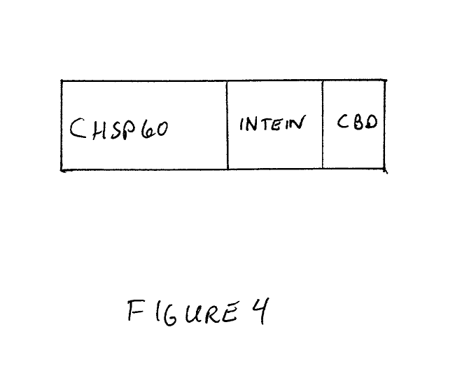

As used herein, CHSP60₁₋₅₄₄ refers to a purified peptide having an amino acid sequence substantially as shown in FIG. 2. CHSP60₁₋₅₄₄ also refers to a purified peptide substantially as shown in FIG. 2 including sequence variations, for example, as shown in FIG. 3, which do not significantly alter the immunoreactivity of the peptide as discussed herein.

Herein described is an expression system for the production of CHSP60₁₋₅₄₄ as described above and a fragment of CHSP60₁₋₅₄₄ consisting of amino acids 80-277 of CHSP60, designated as CHSP60₈₀₋₂₇₇. Specifically, the expression system is arranged so that CHSP60₁₋₅₄₄ or CHSP60₈₀₋₂₇₇ is produced as a fusion protein which can be purified based on the activity or property of the fusion partner, as described below. The fusion protein is then treated such that the fusion partner is cleaved, producing purified, CHSP60₁₋₅₄₄ or CHSP60₈₀₋₂₇₇. As a result of this arrangement, the termini of the peptides are not blocked or masked by the fusion partner, meaning that more antigenic epitopes are available. As will be apparent to one knowledgeable in the art, given the high degree of homology between proteins of the GroE family, it is very difficult to isolate purified native CHSP60 using traditional means, such as, for example, sizing columns, ion exchange columns or even antibody columns. Furthermore, as discussed above, traditional tags used for isolation of recombinant proteins may mask antigenic determinants.As will be appreciated by one knowledgeable in the art, the availability of larger number of epitopes for antibody reactivity may lead to a more sensitive assay. It is also of note that the lack of a fusion partner means that the use of CHSP60₁₋₅₄₄ or CHSP60₈₀₋₂₇₇ as antigens in the ELISA assays as described herein more closely correspond to those presented in vivo. Furthermore, removal of the fusion partner reduces the number of assays required, allowing many more samples to be screened. Also described are PCR primers for generating a DNA fragment encoding the CHSP60₁₋₅₄₄ protein or CHSP60₈₀₋₂₇₇ fragment for subsequent cloning into other expression vectors, as described below.

In one embodiment, described below, the purified CHSP60₁₋₅₄₄ or CHSP60₈₀₋₂₇₇ fragment is used in ELISA assays for screening samples from patients suspected of having chlamydial infections. As discussed herein, these may include patients having, suspected of having or at risk of developing diseases or disorders such as, but by no means limited to chronic pelvic pain, pelvic inflammation disease, tubal infertility, chronic inflammation, ectopic pregnancy, atheriosclerosis, asthma, stroke, Alzheimer's disease, multiple sclerosis, urogenital tract infections, pneumonia, respiratory infections or other chlamydia associated autoimmune diseases. Specifically, the presence of anti-CHSP60 antibody indicates persistent, acute or repeated chlamydial infections which in turn indicates that the patient is at risk of developing complications, as discussed herein. As will be appreciated by one knowledgeable in the art, complications of C. pneumoniae infections can include, for example, cardiovascular diseases (atheriosclerosis, stroke, abdominal aortic aneurysm, etc), pulmonary diseases for example COPD and asthma, as well as neurodegenerative diseases for example Alzheimer's disease and multiple sclerosis.

CHSP60₈₀₋₂₇₇ was previously used along with CHSP60-GST fusion described above to test sera of individuals with scarring trachoma for antibodies against CHSP60. Compared to the GST fusion protein, the fragment showed not only an increase in the number of positive responses in the cases, but also in the controls (Peeling et al 1999 Infect. Dis. Obstet Gynecol. 7:108-9). Thus, this data indicated that CHSP60₈₀₋₂₇₇ was of no value in analyzing individuals with scarring trachoma. However, as discussed below and as shown in Tables 1 and 2, CHSP60₈₀₋₂₇₇ and the CHSP60₁₋₅₄₄ protein show greater sensitivity and lower background compared to the CHSP60-GST fusion in samples of patients with complications of urogenital tract infections or respiratory infections.

As will be appreciated by one knowledgeable in the art, other suitable fusion partners or expression systems which allow for expression and isolation of the native CHSP60₁₋₅₄₄ or CHSP60₈₀₋₂₇₇ may also be used.

The invention provides kits for carrying out the methods of the invention. Accordingly, a variety of kits are provided. In some embodiments, the kits include purified CHSP60₁₋₅₄₄ and/or CHSP60₈₀₋₂₇₇ and/or expression systems for producing same. In some embodiments, the kit may also include ELISA reagents. The kits may be used for detecting antibodies against CHSP60 in patients having, suspected of having or at risk of developing diseases or disorders such as, but by no means limited to, chronic pelvic pain, pelvic inflammation disease, tubal infertility, chronic inflammation, ectopic pregnancy, atheriosclerosis, asthma, stroke, Alzheimer's disease, multiple sclerosis, urogenital tract infections, pneumonia, respiratory infections or other chlamydia associated autoimmune diseases. As will be appreciated by one knowledgeable in the art, the kits may also include instructions for purification and/or preparation of CHSP60₁₋₅₄₄ and/or CHSP60₈₀₋₂₇₇, as described below.

The kits of the invention comprise one or more containers comprising purified CHSP60₁₋₅₄₄ and/or CHSP60₈₀₋₂₇₇ or an expression system for producing same and a set of instructions, generally written instructions although electronic storage media (e.g., magnetic diskette or optical disk) containing instructions are also acceptable, relating to the use intended for the purified peptides or expression system. The containers may contain unit doses, bulk packages (e.g., multi-dose packages) or sub-unit doses.

The purified CHSP60₈₀₋₂₇₇ fragment or CHSP60₁₋₅₄₄ of the kit may be packaged in any convenient, appropriate packaging. For example, if there is a freeze-dried formulation, an ampoule with a resilient stopper is normally used, so that the peptide may be easily reconstituted by injecting fluid through the resilient stopper.

The following Examples are provided to illustrate, but not limit, the invention.

### EXAMPLE I

### Bacterial Isolates

The bacterial isolates used in the present invention were from a laboratory collection. All cultures were grown in Minimal Essential Media supplemented with 10% fetal bovine serum, 2 μM L-glutamine, 25 μg/ml gentamycin, 100 μg/ml vancomycin, and 1 μg/ml cycloheximide.

### EXAMPLE II

### Isolation of Genomic DNA

High molecular weight genomic DNA was isolated by SDS/proteinase K digestion at 56° C. for 3 hours followed by phenol/chloroform extraction and ethanol precipitation. DNA concentration was determined by UV spectroscopy, at A₂₆₀ and purity estimated by the A₂₆₀/A₂₈₀ ratio.

### EXAMPLE III

### PCR Amplification

The deoxyribonucleotides triphosphates dATP, dCTP, dGTP, dTTP are added to the synthesis mixture, either separately or together with the primers, in adequate amounts and the resulting solution is heated to 95° C. for 5 minutes. After this heating period, the solution is subjected to 35 cycles of 1 minute at 95° C., 1 minute at 55° C., 1.5 minutes at 72° C. Following the final cycle, the solution was held at 72° C. for 10 minutes, and then cooled to 4° C. The agent used for the polymerase chain reaction (PCR) was Taq DNA polymerase, purchased from GIBCO Life Technologies.

The following PCR conditions were used: 50 mM KCl, 10 mM Tris, pH 8.4, 1.5 mM MgCl₂, 200 μM of each dNTP (all final concentrations), 50 ng of genomic DNA, 2.5 U of Taq DNA polymerase (GIBCO) and 0.5 μM of each of the degenerate primers MH279 and MH280, and CLH1 and CLH2, described below. A final volume made up to 50 μl with dH₂O was used.

The sequences of the primers used to amplify the CHSP60 protein fragment encompassing amino acids 80-277 from C. trachomatis, C. pneumoniae, and C. psittaci are as follows:

MH279: 5′ AAA ACT CAT ATG AAA GCW GGV GAY GGA ACY ACA ACA 3′
MH280: 5′ CAT AGC TGC TCT TCC GCA WCC RAA VCC WGG AGC TTT MAC WGC 3′

It is of note that these degenerative primers have been used previously (Goh et al, 1996, J Clin Micro 34: 818-823) for generating a DNA fragment used as a species-specific probe for identifying different HSP60 genes.

The sequences of the primers used to amplify the DNA encoding CHSP60₁₋₅₄₄ protein from C. trachomatis, C. pneumoniae, and C. psittaci are as follows:

CLH1: 5′ AGM RCA CAT ATG GYM GCK AAA AAY ATT AAA TAY AA 3′
CLH2: 5′ TWR TWC YGC TCT TCC GCA YTA RTA GTC CAT TCC TGC GCY WG 3′

It is of note that the 5′ end sequences of MH279 and CLH1 contain an Ndel restriction site, while the 3′ end sequences of MH280 and CLH2 contain a Sapl restriction site.

The amplified PCR products were digested with Ndel and Sapl and then ligated into pCYB1. The recombinant plasmids were transformed into competent E. coli and screened and selected.

### EXAMPLE IV

### Plasmid Construction

Escherichia coli JM109 containing the pCYB1 plasmids encoding the CHSP60₁₋₅₄₄ protein and the CHSP60₈₀₋₂₇₇ protein were generated using the New England Biolabs™ IMPACT I kit. Expression and purification of the CHSP60 proteins were performed according to established protocols for the IMPACT I (Intein Mediated Purification with an Affinity Chitin-binding Tag) kit. The IMPACT I system utilizes a protein splicing element, an intein, from Saccharomyces cerevisiae VMA1 gene. The intein has been modified such that it undergoes a self-cleavage reaction at its N-terminus at low temperatures in the presence of thiols such as 1,4-dithiothreitol (DTT), β-mercaptoethanol or cysteine. The gene/nucleic acid encoding the target protein/protein fragment is inserted into a multiple cloning site (mcs) of the pCYB1 vector to create a fusion between the C-terminus of the target gene and the N-terminus of the gene encoding the intein. The DNA encoding a small 5 kDa chitin binding domain (CBD) from Bacillus circulans has been added to the C-terminus of the intein for affinity purification of the 3 part fusion, shown schematically in FIG. 4. When crude extracts of cells from an inducible E. coli expression system are passed through a chitin column, the fusion protein binds to the chitin column while all other contaminants are washed through the column. The fusion is then induced to undergo an intein-mediated self-cleavage on the column by overnight incubation at 4° C. in the presence of DTT or β-mercaptoethanol. The target protein is released while the intein-chitin binding domain fusion partner remains bound to the column.

### EXAMPLE V

### CHSP60 Protein Expression and Purification

A 10 ml culture of LB broth containing 100 μg/ml ampicillin was inoculated with a freshly grown colony of the E. coli clone to be cultured. The culture was then incubated at 37° C. overnight with shaking. The overnight culture was used to inoculate a 1 L flask of LB, which was then grown at 37° C. with shaking to OD₆₀₀ of 0.6-0.8. IPTG was added to the culture to a final concentration of 0.7 mM and the culture was transferred to 30° C. The culture was incubated for a further 3 hours with moderate shaking. The cells were then spun down from the culture at 5000×g for 15 minutes at 4° C. and the supernatant was discarded. The pellet was resuspended in 10 ml of ice-cold Column Buffer (2.42 g Tris-HCl, 29.22 g NaCl, 0.0372 g EDTA, 1 ml Triton X-100 per liter) and the cells were lysed by sonication on ice. The lysed cells were centrifuged at 12,000×g for 30 minutes and the pellet was discarded. The supernatant was loaded onto a chitin column at a rate of approximately 1 drop per second at 4° C. The column was then washed with 200 ml of Column Buffer at a flow rate of one drop per second at 4° C. All traces of the cell extract were washed off the sides of the column. The column was then quickly flushed with 30 ml of Cleavage Buffer (2.42 g Tris-HCl, 2.92 g NaCl, 0.0372 g EDTA per liter) containing 30 mM DTT at 4° C. The column flow was stopped when almost all of the Cleavage Buffer had passed through the column. The column was left at 4° C. overnight. The target protein was eluted from the column using 20 ml of Cleavage Buffer without DTT and 1 ml fractions were collected. The fractions were stored at −20° C. Fractions were analyzed by Bradford assay, SDS-PAGE and Western blotting using anti-CHSP60 antibodies. The eluted fractions were dialyzed against 5 liters of PBS for 4 hours at 4° C. The PBS was replaced and the protein fractions were dialyzed overnight at 4° C.

### EXAMPLE VI

### Enzyme-Linked Immunosorbant Assay

The enzyme-linked immunosorbant assay (ELISA) was performed as follows. One hundred microliters of CHSP60 protein (10 ng) was added to each well of a 96-well microtiter plate and allowed to adsorb for 3 hours at 37° C. or overnight at 4° C. The unbound antigen was washed from the plate and discarded, and the wells were blocked with 150 μl of 3% bovine serum albumin (BSA) in PBS for 90 minutes. The plates were then washed and 100 μl of patient sera (1:500 dilution in PBS containing 0.5% BSA and 0.2% Tween 20) was added and incubated for 60 minutes at 37° C. The wells were then washed three times with PBS containing 0.2% Tween 20, and then 100 μl of horseradish peroxidase-conjugated goat anti-human immunoglobulin antibody (1:4000) was added to each well and incubated for 60 minutes at 37° C. The wells were then washed three times with PBS containing 0.2% Tween 20 and then bound antibody was detected by the addition of 100 μl of substrate (4 mg of 2,2′-azino-bis[3-ethyl-benz-thiazoline-6-sulfonate]/ml in 0.1 M citric acid buffer, pH 4.2 and 10 μl hydrogen peroxide). The plates were then developed in the dark for 20 minutes and the optical density of each well was read in an ELISA reader at 405 nm. All sera were assayed in duplicate against the CHSP60 protein with one negative control serum and 2 positive control sera included with each plate.

To determine the threshold for a positive antibody response, all CHSP60 protein preparations were tested against a panel of 50 Chlamydia-seronegative sera. The threshold is defined as the mean of the response of these negative sera plus three standard deviations from the mean. Results are summarized in Table 1.

Similarly, CHSP60 antibodies in asthma cases and nonasthmatic controls who were Cpn seroreactive were tested for reactivity to CHSP60-GST and CHSP₈₀₋₂₇₇, as shown in Table 2.

### EXAMPLE VII

### CHSP-60 In Atherosclerotic Plaques

Patients undergoing carotid endarectomy for significant symptomatic stenosis (>60%) were enrolled in the study after informed consent. The demographic features and results of immunohistochemical staining for C. pneumoniae, cytomegalovirus and herpes simplex Type I were previously reported (Chiu et al, 1997). Immunohistochemical staining was performed on paraffin embedded sections by the labeled (Strep) avidin-biotin-peroxidase method. The antisera used included C. pneumoniae specific RR402 and CF2 monoclonal antibodies.

Sera were diluted 1:500 and tested against a purified recombinant fragment of CHSP60 (amino acids 80-277) as antigen in a standard ELISA as described previously (Toye et al, 1993, J Infect Dis 168: 1236-1240) and as described above.

Chlamydia serology was performed by the micro-immunofluorescence (MIF) method to detect IgM and IgG antibodies to purified elementary bodies of chlamydia species of C. pneumoniae, C. trachomatis and C. psittaci. (Wang, 1999, in Chlamydia pneumoniae: The Lung and the Heart, Allegra and Blasi, eds, Springer-Verlag Italia: Milano) Sera were screened at 1:16 dilution and all positive sera were titered to end point.

C. pneumoniae antigen was detected by immunohistochemical staining in 54 (72%) of 75 carotid atheromatous plaques. Of the 54 patients with detectable C. pneumoniae antigen, the mean OD was 0.19±0.15 and in the 21 patients without C. pneumoniae antigen the OD was 0.11±0.08, p=0.01 (2 tailed). For CHSP-60 IgG antibody reactivity≧0.12 OD, 38 (70.4%) of 54 patients with C. pneumoniae antigen in atheromas had anti-CHSP-60, versus 5 (23.8%) of 21 patients without C. pneumoniae antigen, p<0.001.

None of the patients had IgM antibodies and 80% of the total cohort had detectable IgG antibodies to C. pneumoniae (>1;16), suggesting a history of C. pneumoniae infection. There was poor correlation between MIF serology and C. pneumoniae antigen detection (previously reported in Chiu et al, 1997). There was also lack of correlation with serology and CHSP-60 antibodies, suggesting that the CHSP60 antibody response is not just a marker of past infection but is uniquely associated with the presence of C. pneumoniae in the atheromas, and possibly with the development of atherosclerosis.

| | C. pneumoniae | C. pneumoniae | |
|---|---|---|---|
| | (Ag Positive) | (Ag Negative) | |
| | (n = 54) | (n = 21) | p value |
| Anti-CHSP-60 | 0.19 ± 0.15 | 0.11 ± 0.08 | 0.01 |
| (Mean OD ± SD) | | | |
| OD ≧ 0.12 | 38 (70.4%) | 5 (23.8%) | <0.001 |

### EXAMPLE VIII

### Discussion

Atherosclerosis involves a low grade chronic inflammatory process (Ross, 1999; Alexander, 1994; Munro and Cotran, 1988), and circulating markers of inflammation such as CRP, fibrinogen, serum amyloid A protein and serum proinflammatory cytokines are predictors of current cardiovascular disease or future myocardial infarction (Danesh et al, 1998, JAMA 279: 1477-1482; Ridker, 1999, Am Intern Med 130: 933-937; Koenig et al, 1999, Circulation 99: 237-242; Kuller et al, 1996, Am J Epid 144: 537-547). It has been postulated that these circulating markers of inflammation may possibly be associated with the presence of infectious agent(s), such as C. pneumoniae, playing a role in atherogenesis. A recent study of patients with coronary artery disease (N=302) and seropositive for C. pneumoniae (≧1:16 IgG titer), demonstrated a reduction in global tests of 4 inflammatory markers (CRP, IL-1, IL-6 and TNFα) 3 months after the completion of a 3 month regimen of azithromycin (Anderson et al, 1999, Circulation 99: 1538-1539). In a study from Finland, part of the 8.5 year trial in the Helsinki Heart Study, the independent and joint effects of infections and inflammation were studied in a nested case: control design (Roivainen et al, 2000, Circulation 101: 252-257). Both C. pneumoniae and herpes simplex virus-I antibodies were associated with increased risk for coronary artery disease.

One of the mechanisms by which C. pneumoniae may be involved in the pathogenesis of atherosclerosis is through chlamydial heat shock protein. It has been postulated that molecular mimicry of CHSP-60 with human HSP60 may induce an autoimmune reaction, leading to activation of inflammatory pathways and an increase in concentration of inflammatory markers (Mayr et al, 1999, Circulation 99: 1560-1566). Specifically, the homology between the amino acid sequence of the 80-277 fragment and the corresponding human HSP60 fragment is 50%. Our results suggest that immune response against epitopes within this region of the chlamydial HSP60 may have elicited an autoimmune response due to cross-reactivity to epitopes of the human HSP-60. It is also of note that patients with C. pneumoniae antigen in atheromatous plaques have significantly higher levels of CHSP-60 antibodies than those without detectable antigen. Furthermore, CHSP-60 localizes in human atheroma and regulates TNFα and matrix metalloprotease expression (Kol et al, 1998), factors that are considered atherogenic. In addition, CHSP-60 is able to activate human vascular endothelium, smooth muscle cells and macrophages (Kol et al, 1999, J Clin Invest 103: 571-577), and in vitro it can stimulate low density lipoprotein (LDL) cellular oxidation (Kalayoglu et al, 1999, J Infect Dis 180: 780-790), the major harmful component of LDL. Recently, it was also been shown that serum antibodies to CHSP-60 cross-react with human HSP-60 and mediate endothelial cytotoxicity, a key event in pathogenesis of atherosclerosis (Mayr et al, 1999).

In summary, CHSP-60 antibody levels are correlated with the presence of C. pneumoniae antigen in atheromas and may play a role in the pathogenesis of atherosclerosis.

While the preferred embodiments of the invention have been described above, it will be recognized and understood that various modifications may be made therein, and the appended claims are intended to cover all such modifications which may fall within the spirit and scope of the invention.

TABLE 1
Absorbance readings at 405 nm for the CHSP60 ELISA assay
comparing the mean ± standard deviation for each serum sample
tested against the Chlamydia trachomatis serovar L₂ CHSP60-GST
fusion protein (whole CHSP60 protein) from Richard Stephens
and the CHSP60 protein fragment from serovar D
(Health Canada protein).

| | Mean ± Standard Deviation | |
|---|---|---|
| | CHSP60-GST | CHSP60 |
| | fusion protein | protein fragment from |
| Sample | (whole protein) | C. trachomatis serovar D |
| PBS (negative control) | 0.001 ± 0.001 | 0.001 ± 0.001 |
| A9302 | 0.090 ± 0.033 | 0.072 ± 0.010 |
| (clinical negative control) | | |
| 7698 | 0.216 ± 0.019 | 0.250 ± 0.001 |
| (low positive control) | | |
| 7710 | 0.633 ± 0.078 | 0.908 ± 0.061 |
| (high positive control) | | |
| St | 0.112 ± 0.005 | 0.065 ± 0.004 |
| 2 | 0.030 ± 0.002 | 0.033 ± 0.001 |
| 9 | 0.076 ± 0.003 | 0.104 ± 0.004 |
| 30 | 0.076 ± 0.023 | 0.063 ± 0.001 |
| 40 | 0.055 ± 0.002 | 0.074 ± 0.001 |
| 47 | 0.069 ± 0.016 | 0.080 ± 0.001 |
| 79 | 0.080 ± 0.003 | 0.082 ± 0.007 |
| 138 | 0.096 ± 0.009 | 0.098 ± 0.002 |
| 214 | 0.053 ± 0.008 | 0.043 ± 0.004 |
| 269 | 0.049 ± 0.002 | 0.051 ± 0.001 |
| 276 | 0.127 ± 0.008 | 0.149 ± 0.022 |
| 357 | 0.158 ± 0.018 | 0.105 ± 0.033 |
| 369 | 0.088 ± 0.008 | 0.089 ± 0.004 |
| 380 | 0.083 ± 0.007 | 0.109 ± 0.002 |
| 428 | 0.047 ± 0.004 | 0.048 ± 0.001 |
| 484 | 0.095 ± 0.001 | 0.080 ± 0.011 |
| 497 | 0.077 ± 0.016 | 0.063 ± 0.013 |
| 560 | 0.109 ± 0.007 | 0.086 ± 0.006 |
| 588 | 0.071 ± 0.012 | 0.075 ± 0.012 |
| 610 | 0.125 ± 0.016 | 0.117 ± 0.008 |
| 620 | 0.064 ± 0.001 | 0.070 ± 0.003 |
| 632 | 0.103 ± 0.001 | 0.096 ± 0.001 |
| 659 | 0.100 ± 0.014 | 0.103 ± 0.008 |
| 680 | 0.259 ± 0.024 | 0.239 ± 0.010 |
| 763 | 0.233 ± 0.008 | 0.299 ± 0.017 |
| 826 | 0.053 ± 0.001 | 0.064 ± 0.004 |
| 840 | 0.063 ± 0.004 | 0.071 ± 0.001 |
| 841 | 0.117 ± 0.030 | 0.106 ± 0.013 |
| 896 | 0.041 ± 0.012 | 0.038 ± 0.008 |
| 963 | 0.084 ± 0.001 | 0.115 ± 0.003 |
| 987 | 0.036 ± 0.007 | 0.031 ± 0.004 |
| 1051 | 0.086 ± 0.001 | 0.066 ± 0.004 |
| 1057 | 0.127 ± 0.004 | 0.160 ± 0.005 |
| 1080 | 0.086 ± 0.006 | 0.070 ± 0.007 |
| 1094 | 0.050 ± 0.011 | 0.054 ± 0.007 |
| 1102 | 0.136 ± 0.034 | 0.116 ± 0.001 |
| 1110 | 0.026 ± 0.005 | 0.025 ± 0.001 |
| 1127 | 0.115 ± 0.004 | 0.202 ± 0.014 |
| 1160 | 0.060 ± 0.008 | 0.069 ± 0.008 |
| 1184 | 0.130 ± 0.033 | 0.097 ± 0.023 |
| 1313 | 0.086 ± 0.001 | 0.131 ± 0.006 |
| 1350 | 0.103 ± 0.011 | 0.117 ± 0.007 |
| 1357 | 0.058 ± 0.001 | 0.076 ± 0.002 |
| 1420 | 0.079 ± 0.001 | 0.059 ± 0.012 |
| 1427 | 0.051 ± 0.001 | 0.060 ± 0.007 |
| 1433 | 0.165 ± 0.003 | 0.141 ± 0.006 |
| 1436 | 0.240 ± 0.008 | 0.285 ± 0.001 |
| 1454 | 0.212 ± 0.029 | 0.270 ± 0.018 |
| 1478 | 0.182 ± 0.001 | 0.197 ± 0.003 |
| 1501 | 0.126 ± 0.008 | 0.107 ± 0.012 |
| 1505 | 0.155 ± 0.007 | 0.139 ± 0.014 |
| 1507 | 0.194 ± 0.025 | 0.265 ± 0.002 |
| 1512 | 0.122 ± 0.004 | 0.136 ± 0.000 |
| 1514 | 0.161 ± 0.006 | 0.135 ± 0.010 |
| 1522 | 0.139 ± 0.013 | 0.098 ± 0.007 |
| 1527 | 0.112 ± 0.008 | 0.088 ± 0.025 |
| 1530 | 0.080 ± 0.004 | 0.097 ± 0.001 |
| 1538 | 0.196 ± 0.017 | 0.223 ± 0.012 |
| 1542 | 0.167 ± 0.008 | 0.222 ± 0.004 |
| 1547 | 0.166 ± 0.003 | 0.153 ± 0.007 |
| 1550 | 0.097 ± 0.008 | 0.110 ± 0.001 |
| 1552 | 0.408 ± 0.035 | 0.143 ± 0.019 |
| 1559 | 0.122 ± 0.021 | 0.147 ± 0.022 |
| 1567 | 0.184 ± 0.024 | 0.191 ± 0.021 |
| 1572 | 0.131 ± 0.006 | 0.132 ± 0.001 |
| 1576 | 0.186 ± 0.023 | 0.203 ± 0.014 |
| 1577 | 0.163 ± 0.013 | 0.190 ± 0.026 |
| 1579 | 0.348 ± 0.093 | 0.265 ± 0.013 |
| i581 | 0.122 ± 0.018 | 0.140 ± 0.003 |
| 1582 | 0.070 ± 0.005 | 0.081 ± 0.007 |
| 1583 | 0.108 ± 0.002 | 0.103 ± 0.005 |
| 1587 | 0.197 ± 0.005 | 0.204 ± 0.007 |
| 1590 | 0.102 ± 0.001 | 0.089 ± 0.004 |
| 1595 | 0.179 ± 0.002 | 0.173 ± 0.000 |
| 1598 | 0.172 ± 0.018 | 0.182 ± 0.014 |
| 1621 | 0.152 ± 0.004 | 0.175 ± 0.010 |
| 1627 | 0.256 ± 0.026 | 0.132 ± 0.004 |
| 1632 | 0.178 ± 0.003 | 0.123 ± 0.011 |
| 1649 | 0.162 ± 0.021 | 0.201 ± 0.013 |
| 1652 | 0.267 ± 0.033 | 0.253 ± 0.028 |
| 1672 | 0.049 ± 0.006 | 0.036 ± 0.001 |
| 1677 | 0.186 ± 0.006 | 0.125 ± 0.001 |
| 1683 | 0.183 ± 0.067 | 0.141 ± 0.008 |
| 1687 | 0.301 ± 0.067 | 0.415 ± 0.045 |
| 1688 | 0.054 ± 0.004 | 0.043 ± 0.003 |
| 1692 | 0.065 ± 0.009 | 0.050 ± 0.008 |
| 1693 | 0.095 ± 0.006 | 0.098 ± 0.000 |
| 1695 | 0.134 ± 0.006 | 0.129 ± 0.025 |

TABLE 2
CHSP60 antibodies versus HSP60 (Cpn Fragment) antibodies
in asthma cases and nonasthmatic controls who were Cpn
seroreactive (MIF IgG 1:16)

| STUDY GROUP | No. | Findings | |
|---|---|---|---|
| All subjects | 143 | | |
| CHSP60 v HSP60 (Cpn) | | r = 0.012, p = .889 | Pearson |
| | | r = 0.068, p = .356 | Spearman |
| CHSP60 v FEV1/FVC % pred | | r = 0.17, p = .13 | Pearson |
| HSP60 (Cpn Fragment) v | | r = .24, p = .02 | Pearson |
| FEV1/FVC % pred | | | |
| CHSP60 (Gst fusion) | | O.D. × 1000 (Sd) | P-value* |
| ASTHMA | 91 | 131 (207) | |
| CONTROLS | 52 | 94 (154) | .82 |
| HSP60 (Cpn Frag) | | O.D. × 1000 (Sd) | P-value* |
| ASTHMA | 91 | 156 (115) | |
| CONTROLS | 52 | 114 (57) | .03 |

*by ANOVA (log-transformed data) controlled for age, sex and (ever) smoking

1. A method of detecting anti-CHSP60 antibodies in a sample from a patient comprising:
providing purified CHSP60₈₀₋₂₇₇ or purified CHSP60₁₋₅₄₄;
binding the CHSP60₈₀₋₂₇₇ or CHSP60₁₋₅₄₄ to a support;
mixing the sample with the bound CHSP60₁₋₅₄₄ or CHSP60₈₀₋₂₇₇ under conditions such that anti-CHSP60 antibodies within the sample bind to the CHSP60₁₋₅₄₄ or CHSP60₈₀₋₂₇₇;
washing away unbound sample; and
detecting antibodies bound to the CHSP60₁₋₅₄₄ or CHSP60₈₀₋₂₇₇.

2. The method according to claim 1 wherein the purified CHSP60₁₋₅₄₄ or CHSP60₈₀₋₂₇₇ is provided by:
expressing CHSP60₁₋₅₄₄ or CHSP60₈₀₋₂₇₇ as a cleavable fusion protein having a fusion partner in a suitable host;
isolating the cleavable fusion protein;
cleaving the fusion partner from the fusion protein; and
recovering purified CHSP60₁₋₅₄₄ or CHSP60₈₀₋₂₇₇.

3. The method according to claim 1 wherein the serum is from a patient having a urogenital tract infection or a respiratory infection.

4. A kit comprising purified CHSP60₈₀₋₂₇₇.

5. The kit according to claim 4 further comprising purified CHSP60₁₋₅₄₄.
